(19) United States
(12) Patent Application Publication
(10) Pub. No.: US 2010/0248224 A1
(43) Pub. Date: Sep. 30, 2010

(54) DNA FRAGMENTATION ASSAY

(75) Inventors: Tina Garyantes, Warren, NJ (US); Zhuyin Li, Flemington, NJ (US); Yongping Yan, Bridgewater, NJ (US); Justin Anthony Yu, Chalfont, PA (US); Asher Zilberstein, Doylestown, PA (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/303,588

(22) PCT Filed: Jun. 20, 2007
(86) PCT No.: PCT/US07/71619

§ 371 Date: Apr. 28, 2010

Related U.S. Application Data
(60) Provisional application No. 60/805,409, filed on Jun. 21, 2006.

(51) Int. Cl. C12Q 1/68

(52) U.S. Cl. 435/6

(57) ABSTRACT

The present invention provides methods for the detection of agents that modify the formation of DNA fragmentation in cells. The disclosed methods are configured in an assay format amendable to high throughput screening applications.

### BACKGROUND OF THE INVENTION

In normally functioning biological systems, cell number is regulated by the balance between cell proliferation and apoptosis. An inappropriate balance between cell proliferation and apoptosis has been implicated in the etiology of many diseases. For instance, an exacerbation of apoptotic mechanisms is thought to contribute to neurodegenerative diseases such as Alzheimer's disease, autoimmune diseases exemplified by Multiple Sclerosis and ischemia-associated injuries such as stroke. Conversely, a mitigation of appropriate apoptotic pathways is thought to be an underlying mechanism of diseases such as cancer.

Apoptosis is characterized by several hallmark features including cell shrinkage and cytoplasmic membrane blebbing, chromatin condensation, nuclear DNA fragmentation and protein degradation. There are at least two distinguishable apoptotic pathways—the extrinsic and intrinsic pathways (for review, see Oncogene 23:2861-2874, 2004; Photochem. Photobiol. Sci. 3:721-729, 2004). The extrinsic or receptor-mediated pathway is induced by death receptor ligands, such as tumor necrosis factor. The death receptor ligands signal through the caspase cascade, ultimately resulting in nuclear DNA digestion by caspase activated nucleases. The intrinsic pathway, signaling through mitochondrial mechanisms, is sensitive to environmental stressors like ultraviolet light or drugs. These stresses cause permealization of the mitochondrial membrane leading to the release of cytochrome c, endonuclease G, apoptosis inducing factor (AIF) as well as many other unidentified molecules. The relative contribution of the extrinsic or intrinsic pathway to apoptosis is determined by the balance between proapoptotic and cell survival factors. (for review, see J. Intern. Med. 258:479-517, 2005).

Apoptosis can be induced through death receptor ligand binding, activation of apoptosis inducers, activation of caspases, down regulation of cell survival molecules, or through other known and unknown novel mechanisms. These all lead to DNA fragmentation, thus, a phenotypic DNA fragmentation assay will identify all apoptosis-inducing compounds irrespective of mode of action and potentially identify compounds with novel mechanisms. A gel-based DNA ladder assay is the gold standard assay for DNA fragmentation. However, the labor-intensive and multi-step nature of the gel-based DNA ladder assay is not amenable to high-throughput screening efforts. Thus, there is a need for a screening assay that would identify agents acting through the classical apoptosis pathways and novel mechanisms as well. Cytotoxic assays could potentially be employed but these assays are non-selective in that they identify compounds involved in both apoptosis- and nonapoptosis-mediated cell death and can lead to significant false positives. A non-radioactive and robust assay that is amenable to high-throughput screening would be preferred.

Currently, three different formats have been utilized for screening of compounds involved in apoptosis. The first one is based on a radiometric filtration method, where cells are grown in 3H-thymidine and then intact DNA is separated from fragmented DNA using a glass-fiber filter plate (Anal. Biochem. 242:187-196, 1996). The throughput of the radiometric assay is limited by the hazard associated with large amounts of radioactivity and the laborious nature of the assay. The second assay format is a TUNEL assay which is based on labeling of 3′ double-stranded DNA (dsDNA) with fluorescent-dUTP by a transferase enzyme and then detection by flow cytometry or imaging methods. There are many TUNEL assay kits available but all of them are labor intensive and only a few samples can be tested per assay. The third assay format is a sandwich ELISA assay using anti-DNA and anti-histone antibodies. This assay is also labor intensive and the need for two antibodies makes it relatively costly for high-throughput compound screening.

An efficient and nonradioactive assay format would be to employ a DNA intercalator such as PicoGreen or propidium iodide to detect fragmented DNA. For example, PicoGreen is a small organic molecule that intercalates into the major groove of dsDNA. PicoGreen has been a useful tool to study DNA levels in blood samples (Scan. J. Immunol. 57:525-533, 2003; Clin. Immunol. 106:139-147, 2003; Blood 102(6):2243-2250, 2003). Using DNA intercalators, the present invention provides methods for the detection of agents that modify formation of DNA fragments in cells and is amendable to high throughput screening applications.

### BRIEF DESCRIPTION OF THE DRAWINGS

FIG. 1 shows the change in PicoGreen fluorescence in relative fluorescence units (RFU) in HL-60 lysates following treatment of cells with camptothecin (campto) or DMSO (control). (RFU, relative fluorescence units; DMSO, dimethyl sulfoxide)

FIG. 2 shows the PicoGreen fluorescence signal (RFU, relative fluorescence units) is dependent on the level of DNA in the cell lysates following treatment of HL-60 cells with camptothecin (campto). (RFU, relative fluorescence units)

FIG. 3 shows the effects of selected compounds such as camptothecin (campto), staurosporin and bleomycin on DNA fragmentation in HL-60 cells as detected by PicoGreen. (RFU, relative fluorescence units)

FIG. 4 shows the effects of camptothecin (campto) on DNA fragmentation in HL-60 cells as detected by propidium iodide. (RFU, relative fluorescence units)

FIG. 5 shows the effects of camptothecin (campto), staurosporin and bleomycin on DNA fragmentation in HL-60 cells as detected by ELISA. (Abs, absorbance)

FIG. 6 shows the effect of an apoptosis inhibitor, ZnCl₂, on camptothecin-induced DNA fragmentation as detected by PicoGreen. (RFU, relative fluorescence units)

FIG. 7 shows that RNase treatment improves the DNA fragmentation signal to background ratio in HL-60 cell lysates as detected by PicoGreen. (RFU, relative fluorescence units)

FIG. 8 shows the time course of camptothecin (campto) effects on DNA fragmentation detected by PicoGreen in HL-60 lysates. (RFU, relative fluorescence units; hr, hour; DMSO, dimethyl sulfoxide)

FIG. 9 shows the effect of HL-60 cell density on DNA fragmentation detected by PicoGreen in HL-60 lysates. (RFU, relative fluorescence units; DMSO, dimethyl sulfoxide)

FIG. 10 shows the fold-induction in PicoGreen DNA fragmentation signal in relation to HL-60 cell density.

FIG. 11 shows the effect of DMSO concentrations on HL-60 cells. (RFU, relative fluorescence units; DMSO, dimethyl sulfoxide)

FIG. 12 shows the induction of DNA fragmentation detected by PicoGreen following incubation of HL-60 cells with valinomycin, vinblastine or vincristine.

FIG. 13 shows the induction of DNA fragmentation detected by PicoGreen following incubation of HL-60 cells with etoposide, genistein, puromycin or rapamycin.

FIG. 14 shows the data distribution of screening a random chemical library using PicoGreen detection of DNA fragmentation in HL-60 lysates. (RFU, relative fluorescence units)

### DETAILED DESCRIPTION OF THE INVENTION

All publications cited herein are hereby incorporated by reference. Unless defined otherwise, all technical and scientific terms used herein have the same meaning as commonly understood to one of ordinary skill in the art to which this invention pertains.

The terminology used in this specification and the appended claims is for the purpose of describing particular embodiments only and use in the specification is not intended to be limiting of the invention. The singular forms of a word are intended to include the plural forms unless the context clearly indicates otherwise. For example, the singular forms of “a”, “an” and “the” are intended to include the plural forms as well. Further, reference to an agent May include a mixture of two or more agents. Thus, the term “an agent” includes a plurality of agents, including mixtures and/or enantiomers thereof. It should also be noted that the term “or” is generally employed in its sense including “and/or” unless the content clearly dictates otherwise. It will be further understood that the terms “comprises” and/or “comprising,” when used in this specification, specify the presence of stated features, steps, elements, and/or components, but do not preclude the presence or addition of one or more other features, steps, elements, components, and/or groups thereof.

Furthermore, in accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Thus, an embodiment of the invention is a method of identifying an agent that modifies the formation of DNA fragments, the method comprising: (a) providing cells in an array of receptacles; (b) adding an agent to at least one receptacle; (c) incubating the agent with the cells for a predetermined period of time; (d) lysing the cells; (e) adding a detectable compound capable of intercalating into DNA fragments to said at least one receptacle; (f) measuring the amount of detectable compound intercalated; and (g) comparing the amount of intercalated detectable compound to a control to determine a difference thereby identifying said agent as a modifying agent when the difference exceeds a predetermined threshold.

The assay detects DNA fragments. The DNA fragments may be small double-stranded DNA (dsDNA) fragments in the cytoplasmic fraction of cell lysates and dsDNA fragments released from apoptotic cells into the medium. Further, the DNA fragments may be single-stranded DNA (ssDNA) fragments in the cytoplasmic fraction of cell lysates and ssDNA fragments released from apoptotic cells into the medium. In an embodiment of the invention, the assay is utilized to measure spontaneous apoptosis such as, but not limited to, apoptosis during co-culture in the presence or absence of different cell types or different culturing conditions. Thus, DNA fragment formation may be induced by removing an ingredient from the culture medium such as fetal bovine serum. Another embodiment of the invention utilizes the assay to measure the absence of apoptosis. In another embodiment, the presence or increase of apoptosis or the absence or decrease of apoptosis can be measured during treatment of cells with an agent. In a further embodiment of the invention, the assay is utilized to measure cell survival or cell proliferation.

A further embodiment of the invention is a method of identifying an agent that modifies the formation of DNA fragments, the method comprising: (a) providing cells in an array of receptacles; (b) adding to at least one receptacle a component selected from the group consisting of an inducer, an inhibitor, a modulator, a modulator of the inducer and a modulator of the inhibitor; (c) incubating the component with the cells for a predetermined period of time; (d) adding an agent to said at least one receptacle; (e) incubating the agent with the cells for a predetermined period of time; (f) lysing the cells; (g) adding a detectable compound capable of intercalating into DNA fragments to said at least one receptacle; (h) measuring the amount of detectable compound intercalated; and (i) comparing the amount of intercalated detectable compound to a control to determine a difference thereby identifying said agent as a modifying agent when the difference exceeds a predetermined threshold.

Refinements such as adding the inducer, the inhibitor or the modulator with the agent in a single step are well within the knowledge and capability of the skilled artisan and are considered embodiments of the invention.

An embodiment of the invention is a component that modifies the formation of DNA fragments by affecting apoptosis, cell survival or cell proliferation. The component is selected from the group consisting of an inducer, an inhibitor, a modulator, a modulator of the inducer and a modulator of the inhibitor.

Cell survival is the ability of a cell to stay alive in favorable or unfavorable conditions. Unfavorable conditions include but are not limited to the presence of one or more toxic compounds, nutrient deprivation, or lack of oxygen. As a non-limiting example, some cancer cells have increased expression of survival proteins, for example Bcl2, which make the cells resistant to apoptosis. Others cancer cells have developed mechanisms which make the cells survive better or be less prone to apoptosis under conditions of low oxygen level.

Cell proliferation is an increase in cell number. Non-limiting examples of cell proliferation are an increase in cell number due to normal cell division, an induction of cell division or an inhibition of cell death.

An embodiment of the invention is a method of identifying an agent that modifies the formation of DNA fragments by cell undergoing apoptosis. However, the invention is not limited to any particular form of cell death. The invention can be applied to any mechanism of cell death where DNA fragmentation is a terminal event.

The term “inhibitor” encompasses any drug, chemical, protein or protein fragment capable of blocking, interrupting or preventing a cellular response, activity or pathway involved in apoptosis, cell survival or cell proliferation. Further, an inhibitor may be, e.g., a molecular chaperone, antibody or inhibitory RNA (RNAi) that blocks expression of cellular proteins thereby inhibiting pathways directly or indirectly. An “inhibitor” may be the manipulation of culturing conditions such as oxygen augmentation or deprivation or changing media components in such a manner as to block, interrupt or prevent a cellular response.

The term “inducer” encompasses any drug, chemical, protein or protein fragment capable of initiating or stimulating a cellular response, activity or pathway involved in apoptosis, cell survival or cell proliferation. Further, an inducer may be a molecular chaperone, antibody or inhibitory RNA (RNAi) that blocks expression of cellular proteins thereby removing inhibition or directly initiating or stimulating a cellular response, activity or pathway. An “inducer” may be the manipulation of culturing conditions such as oxygen augmentation or deprivation or changing media components in such a manner as to block, interrupt or prevent a cellular response.

The term “modulator” encompasses any drug, chemical, protein or protein fragment capable of adjusting the intensity, proportion or the characteristics of a cellular response, activity or pathway involved in apoptosis, cell survival or cell proliferation. Further, a modulator may be a molecular chaperone, antibody or inhibitory RNA (RNAi) that blocks expression of cellular proteins thereby removing inhibition or inducing a cellular response, activity or pathway. A “modulator” may be the manipulation of culturing conditions such as oxygen augmentation or deprivation or changing media components in such a manner as to block, interrupt or prevent a cellular response.

in a further embodiment of the invention, a modulator may be used in conjunction with an inducer such that a modulator of an inducer makes the inducer more potent (e.g., resulting in an enhanced cellular response) or the inducer less potent (e.g., resulting in a reduced cellular response). A modulator of an inhibitor makes the inhibitor less potent (e.g., enhanced cellular response) or the inhibitor more potent (e.g., reduced cellular response).

Inhibitors, inducers or modulators can be utilized to mimic pathways or aspects of disease states. As a non-limiting illustrative example, an embodiment of the invention would be to induce apoptosis with β-amyloid fragments to mimic aspects of Alzheimer's disease in the presence or absence of potential modulators such as inflammatory cytokines. A further illustrative example, an embodiment of the invention is to identify agents that promote apoptosis in one or multiple aspects of cancer. Using such a paradigm, a contemplated embodiment of the invention is to quantify the ability of a test agent to induce or enhance apoptosis in cancer cells, tissues or organs. An inducer of apoptosis can be broad acting encompassing many pathways leading to cell death. Alternatively, an inducer of apoptosis can be very specific to a single apoptotic pathway or limited to treating a specific disease or pathological condition. Thus, an embodiment of the invention encompasses a screening method for identifying a test agent that may ameliorate a disease state where apoptosis is thought to be inhibited, for example, cancer. Such cancers include, but are not limited to, acute myeloid leukemia, multiple myeloma, non-Hodgkin lymphoma, chronic lymphocytic leukemia and solid tumors.

Another embodiment of the invention is the use of more than one inducer, inhibitor or modulator. Using more than one inducer, inhibitor or modulator could, but is not limited to, having additive effects, counter effects, synergistic effects or affecting multiple pathways.

An embodiment of the invention utilizes an intercalating detectable compound. A non-limiting example is an intercalating fluorescent dye. Intercalators commonly are heteroaromatic polycyclic molecules that insert between two base pairs in a DNA duplex. However, the invention is not limited to heteroaromatic polycyclic molecules. Any intercalating molecule that shows a significant fluorescent enhancement or shift in emission or excitation parameter(s) in the presence of DNA fragments with little or no nonselective binding to RNA or proteins is contemplated by the present invention. Such intercalating dyes are known to those skilled in the art and include, but are not limited to, the bisbenzimide dye Hoechst 33258. Another useful intercalating detectable compound is propidium iodide. An embodiment of the invention utilizes PicoGreen. PicoGreen belongs to the family of unsymmetric monomethine cyanine dyes. It exhibits high binding constants with DNA and is highly fluorescent when bound to DNA, while virtually non-fluorescent when free in solution. A further embodiment of the invention uses propidium iodide. Further, some intercalators are capable of binding to ssDNA such as TOTO (Nucleic Acids Res. 23:1215-1222, 1995) and OliGreen (Molecular Probes, Cat. #07582, Cat. #011492). An embodiment of the invention utilizes cell-permeant DNA probes such as BENA435 (Nucleic Acids Res. 34:) and thus may eliminate the need to lyse the cells in order to label the DNA. A further embodiment of the invention utilizes YOPRO, Hoechst 33342, DAPI and DRAQ5.

The intercalating detectable molecule is not limited to a fluorescent dye. The amount of DNA fragment can be quantified using any methodology known to those skilled in the art. The amount of intercalating molecules incorporated into DNA can be quantified by labels such as, but not limited to, radioisotopes or scintillant-activating compounds. Detection methods include, but are not limited to, a peptide tag, enzymatic activity, absorbance, fluorescence, time-resolved fluorescence, polarized fluorescence, fluorescence resonance energy transfer, luminescence, bioluminescence resonance energy transfer, radioactive labeling and scintillation proximity or other methods commonly used in the field. In another embodiment, indirect labeling methods may be used including, but not limited to, using labeled antibodies, using streptavidin-biotin interactions, metal chelating affinity reagents or GST-glutathione affinity reagents. Any direct or indirect labeling method known to those skilled in the art is contemplated as part of this invention. In a further embodiment, the amount of DNA can be quantified by the determination of absorbance at 260 nm (A260).

A further embodiment of the invention is to separate chromosomal DNA from DNA fragments before measuring the amount of detectable compound that has intercalated. Separation of chromosomal DNA from DNA fragments May be performed by methods known in the art. Non-limiting examples include centrifugation, filtration, sedimentation, electrophoresis, size-exclusion, affinity purification and precipitation. Any method of separation may be employed by one skilled in the art to separate or remove chromosomal DNA from the DNA fragments.

An embodiment of the invention involves lysing the cells. Cells can be lysed by the addition of a detergent containing lysis buffer. However, the invention is not limited to the use of detergent in the lysis buffer but may include any method that is appropriate for lysing cells. For example, cells may be lysed by exposure to hypotonic buffer, sonication or freeze/thaw. Other methods of lysing cells are well known to those skilled in the art.

A further embodiment of the invention utilizes DNase free RNase to remove RNA in the cell lysates for the purpose of increasing signal to background ratio by reducing background fluorescent signal due to endogenous cellular RNA.

An embodiment of the invention comprises an array of receptacles that can receive cells and other materials such as culture media. An array of receptacles can be any number of receptacles from at least one or more than one receptacle suitable for holding cells within the scope of the invention. Examples include but are not limited to flasks, culture dishes, tubes such as 1.5 ml tubes, 12 well plates, 96 well plates, 384 well plates and miniaturized microtiter plates with perhaps 4000 receptacles (U.S. Patent Application 20050255580). The array of receptacles may be amendable to the addition of a protective covering thus preventing against entry of contaminants or evaporation of contents.

A further characteristic of the receptacles is that the receptacle may allow for analysis, non-limiting examples include, spectrophotometric analysis, scintillation counting and fluorescence measurements. However, this is not a limitation to receptacles that can be used within the scope of the invention given that samples can be transferred to a suitable container amendable for further analysis. A non limiting example is to modify the method such that the method further comprises providing a second array of receptacles wherein the step of lysing the cells further comprises separating supernatant from cell debris and the next step further comprises adding a detectable compound capable of intercalating into DNA fragments to at least one receptacle of said second array of receptacles containing a sample of said separated supernatant.

An embodiment of the invention uses a control. A control is a term of art well understood by skilled artisans. An appropriate control may be dependent on the assay parameters utilized or the experimental question under investigation. A control may be a particular set of assay conditions or the addition or elimination of a particular compound to the culture medium. A control may be considered a positive control in that the assay conditions or control compound added brings about the anticipated response. For example, if the agent under investigation is expected to induce apoptosis, a positive control would be a compound known to induce apoptosis. A non-limiting example of a positive control is the addition of vinblastine sulfate. A control may also be a negative control. A negative control may be a particular set of assay conditions or the addition or elimination of a particular compound to the culture medium that would bring about the anticipated response. For example, if the agent under investigation is expected to induce apoptosis, then a negative control would be expected to not induce apoptosis. A control may be a “vehicle” control. For example, if the test agent is dissolved in DMSO then the vehicle control would be DMSO without test agent. A control may simply be the use of historical data.

An embodiment of the invention uses cell lines that are commercially available. For example, cells that can be used are available from the American Tissue Culture Company. In one embodiment, HL-60 Cells are used. Cells may be prokaryotic or eukaryotic. The invention is not limited by the type of cells used. Primary cultures may also be utilized. Non-differentiated cells may be subjected to various agents to cause the cells to differentiate into a particular phenotype. For example, progenitor cells induced to differentiate into oligodendrocytes would be an embodiment of the invention. The particular cell type used may be selected by markers specifically expressed by the desired cell type, or alternatively, by the loss of a particular marker(s). Cells can be separated or sorted by methods such as flow cytometry that are commonly used by skilled artisans.

An embodiment of the invention uses a homogeneous cell population. An alternative embodiment of the invention uses a heterogeneous cell population. The cells can be of any type and in any proportion to complete the assay of the invention.

Cells may be obtained from a biological sample. A biological sample may include, but is not limited to, tissue or fluids, sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample can be obtained from a eukaryotic organism, including from mammals such as a primate, e.g., chimpanzee, macaque or human, cow, dog, cat, a rodent, e.g., guinea pig, rat, mouse, rabbit, or a bird, reptile, or fish.

Another embodiment of the invention is to use cells transiently or stably transformed to overexpress or not express at least one protein and determine if such expression or lack thereof affects DNA fragmentation. Expression can be induced or constitutive. Agents can be tested for their ability to modulate DNA fragmentation in the transformed cells. Further, test agents can be tested for their ability to modulate DNA fragmentation in transfected cells in the presence or absence of inducers or inhibitors of apoptosis. Such an embodiment may constitute a control.

A recombinant expression vector of the invention comprises a nucleic acid molecule in a form suitable for expression of the nucleic acid in a host cell. Thus, a recombinant expression vector of the present invention can include one or more regulatory sequences, selected on the basis of the host cells to be used for expression, that is operably linked to the nucleic acid to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example. Goeddel, Gene Expression Technology: Methods in Enzymology Vol. 185, Academic Press, San Diego, Calif. (1990). Regulatory sequences include those that direct constitutive expression of the nucleotide sequence in many types of host cells (e.g., tissue specific regulatory sequences). It will be appreciated by those skilled in the art that the design of the expression vector can depend on such factors as the choice of host cell to be transformed, the level of expression of protein desired, etc. The expression vectors of the invention can be introduced into host cells to produce proteins or peptides encoded by nucleic acids as described herein.

The term “overexpression” as used herein, refers to the expression of a polypeptide, e.g., a molecule that may be involved in apoptosis or cell survival mechanisms, by a cell, at a level that is greater than the normal level of expression of the polypeptide in a cell that normally expresses the polypeptide or in a cell that does not normally express the polypeptide. For example, expression of the polypeptide may by 10%, 20%, 30%, 40%, 50%, 60%, 70, 80%, 90%, 100%, or more as compared to expression of the polypeptide in a wild-type cell that normally expresses the polypeptide. Mutants, variants, or analogs of the polypeptide of interest may be overexpressed.

As used herein, the term “transient” expression refers to expression of exogenous nucleic acid molecule(s) which are separate from the chromosomes of the cell. Transient expression generally reaches its maximum 2-3 days after introduction of the exogenous nucleic acid and subsequently declines.

As used here, the term “stable” expression refers to expression of exogenous nucleic acid molecule(s) that are part of the chromosomes of the cell. In general, vectors for stable expression of genes include one or more selection markers.

Cell culturing techniques for transformed, non-transformed, primary culture and biological samples are well known in the art. Biological samples or cultured cells can be stored until required for use. The media used for culturing can be specifically designed or purchased from commercial sources.

The present invention provides methods for identifying (e.g., screening, detecting, characterizing, analyzing and quantifying) agents that modulate the formation of dSDNA or ssDNA fragments. The term “agent”, “test agent”, “test compound”, “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid (e.g., a sphingolipid), fatty acid, polynucleotide, oligonucleotide, etc., which is employed in the assays of the invention and assayed for its ability to modulate DNA fragmentation or apoptosis. There are no particular restrictions as to the compound that can be assayed. Examples include single agents or libraries of small, medium or high molecular weight chemical molecules, purified proteins, expression products of gene libraries, synthetic peptide libraries, cell extracts and culture supernatants. An agent encompasses any combination of different agents.

An agent may include at least one or more soluble and insoluble factors, cell matrix components, conditioned media, cell extracts, tissue extracts, explants, pH modifiers, gasses, osmotic pressure modifiers, ionic strength modifiers, viruses, DNA, RNA or gene fragments. An agent can be in the form of a library of test agents, such as a combinatorial or randomized library that provides a sufficient range of diversity or conversely are limited to similar structures or features. Agents can be optionally linked to a fusion partner, e.g., targeting compounds, rescue compounds, dimerization compounds, stabilizing compounds, addressable compounds, and other functional moieties. Conventionally, new chemical entities with useful properties are generated by identifying a test agent (called a “lead compound” or a “lead”) with some desirable property or activity, e.g., inhibiting activity or modulating activity. The lead compound is then used as a scaffold to create variants of the lead compound, and further evaluate the property and activity of those variant compounds.

An agent may include treatment conditions and manipulation of external and internal conditions or environment. A non-limiting example of such an agent includes ultraviolet light.

An embodiment of the invention is use in high throughput screening (HTS) methods. HTS is the automated, simultaneous testing of thousands of distinct chemical compounds in assays designed to model biological mechanisms or aspects of disease pathologies. More than one compound, e.g., a plurality of compounds, can be tested simultaneously, e.g., in one batch. In one embodiment, the term HTS screening method refers to assays which test the ability of one compound or a plurality of compounds to influence the readout of choice.

Liquid handling systems, analytical equipment such as fluorescence readers or scintillation counters and robotics for cell culture and sample manipulation are well known in the art. Mechanical systems such as robotic arms or “cherry-picking” devices are available to the skilled artisan. Commercial plate readers are available to analyze conventional 96-well or 384-well plates. Single sample, multiple sample or plate sample readers are available that analyze predetermined wells and generate raw data reports. The raw data can be transformed and presented in a variety of ways.

An embodiment of the invention is an assay system for identifying an agent that modulates the formation of double-stranded DNA fragments, the assay system comprising: (a) an array of receptacles; (b) lysis buffer; (c) a detectable compound capable of intercalating into double-stranded DNA; and (d) at least one component wherein the component is selected from the group consisting of the agent(s), inducer(s) of apoptosis, inhibitor(s) of apoptosis, control(s) and cells.

A further embodiment of the invention is a kit comprising at least one element of the assay system and instructions for use. Thus, the components of the assay system may be provided separately or may be provided together such as in a kit. Components of the assay system may be prepared and included in a kit according to methods that maximize the stability of the individual components. Such methods are familiar to those persons skilled in the art. For example, cells of the assay system may be provided as a suspension or lyophilized. Additional components of the system may also be included such as buffers, containers for mixing the assay components such as microtiter plates or test tubes. The assay system can be provided in the form of a kit that includes instructions for performing the assay and instructions for data handling and interpretation.

An embodiment of the invention is a pharmaceutical composition for the modulation of DNA fragment formation comprising as therapeutically effective amount of an agent identified by the methods of the invention and a pharmaceutically acceptable carrier.

The term “therapeutically effective amount” refers to an amount of an agent effective to treat a disease or disorder in a subject or mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The example to cancer is non-limiting since an agent the modulates the formation of dsDNA or ssDNA fragments would have applications to many varied diseases.

A pharmaceutical composition for the modulation of DNA fragment formation may comprise a therapeutically effective amount of an agent wherein the agent modulates DNA fragment formation via a receptor protein. Thus, the pharmaceutical composition may comprise a test agent that is an agonist, a partial agonist, an antagonist or an inverse agonist. Further, the pharmaceutical composition may comprise a test agent that is a peptide, peptide fragments thereof, cognates, congeners, mimics, analogs, or secreting cells and soluble molecules thereof. A further embodiment of the invention is a pharmaceutical composition for the modulation of DNA fragment formation comprising a therapeutically effective amount of the identified agent and a pharmaceutically acceptable carrier, wherein the pharmaceutical composition effectively modulates an apoptotic pathway or mechanism.

As used herein, the term “agonist” refers to moieties (e.g., but not limited to ligands to and agents) that activate the intracellular response when bound to the receptor, or enhance GTP binding to membranes.

As used herein, the term “partial agonist” refers to moieties (e.g., but not limited to, ligands and agents) that activate the intracellular response when bound to the receptor to a lesser degree/extent than do agonists, or enhance GTP binding to membranes to a lesser degree/extent than do agonists.

As used herein, the term “antagonist” refers to moieties (e.g., but not limited to, ligands and agents) that competitively bind to the receptor at the same site as does an agonist. However, an antagonist does not activate the intracellular response initiated by the active form of the receptor and thereby can inhibit the intracellular responses by agonists or partial agonists. In a related aspect, antagonists do not diminish the baseline intracellular response in the absence of an agonist or partial agonist.

As used herein, the term “inverse agonist” refers to moieties (e.g., but not limited to, ligand and agent) that bind to a constitutively active receptor and inhibit the baseline intracellular response. The baseline response is initiated by the active form of the receptor below the normal base level of activity that is observed in the absence of agonists or partial agonists, or decrease of GTP binding to membranes.

As used herein, the term “ligand” refers to a moiety that binds to another molecule, wherein the moiety includes, but certainly is not limited to a hormone or a neurotransmitter, and further refers to ligands wherein the moiety stereoselectively binds to a receptor.

The pharmaceutical compositions of the present invention can be used in combination with other therapeutic agents. For example, in the treatment of cancer, the pharmaceutical composition may be given in combination with cytokines or various chemotherapeutic compounds.

A further embodiment of the invention is a method of diagnosing or monitoring a treatment of a disease wherein a biomarker for the disease comprises the formation of DNA fragments, the method comprising: (a) providing a biological sample in an array of receptacles; (b) adding a detectable compound capable of intercalating into DNA fragments to at least one receptacle; (c) measuring the amount of detectable compound intercalated; and (d) comparing the amount of intercalated detectable compound to a reference to determine a difference thereby diagnosing or monitoring the treatment of the disease when the difference exceeds a predetermined threshold.

A biomarker is a term well known to one skilled in the art. A non-limiting example is the use of the term biomarker to encompass any physiological response, phenotype or characteristic that can be used to quantitate or qualitatively indicate a specific state of the cell, organism and mammal.

A biomarker is considered useful for aiding in the diagnosis, monitoring, and prediction of disease or in monitoring the treatment of a disease when it is significantly different between the subsets of biological samples tested. Levels of a biomarker are “significantly different” when the probability that the particular biomarker has been identified by chance is less than a predetermined value. The method of calculating such probability will depend on the exact method utilized to compare the levels between the subsets, such as t test or similar statistical analysis. As will be understood by those in the art, the predetermined threshold will vary depending on the number of samples utilized.

A biological sample may be organ samples derived from organs of non-human animals or humans, tissue samples derived from tissues of non-human animals or humans, as well as cell samples, derived from cells of non-human animals or humans or from cell cultures. For animal experimentation, biological samples comprise target organ tissues obtained after necropsy or biopsy and body fluids, such as blood. For clinical use of the biomarkers, particular preferred samples comprise body fluids, like blood, sera, plasma, urine, synovial fluid, spinal fluid, cerebrospinal fluid, semen or lymph, as well as body tissues obtained by biopsy.

A reference is understood by one skilled in the art. A reference can include, but is not limited to, a biological sample from a non-diseased subject wherein the subject is a non-human animal or human. Further, a reference can be a biological sample from a non-treated subject. Alternatively, a reference can be from the same subject before, during and after treatment. A reference can be from the same subject but can be a different cell, tissue or organ sample than cell, tissue or organ source used to measure the biomarker. A reference does not have to be a biological sample but can be a sample with a known amount of DNA fragments.

The invention is further described in the following examples, which do not limit the scope of the invention described in the claims. While the invention has been described and exemplified in sufficient detail for those skilled in this art to produce and use it, various alternatives, modifications, and improvements should be apparent without departing from the spirit and scope of the invention. One skilled in the art readily appreciates that the present invention is well adapted to carry out the objective and obtain the ends and advantages mentioned, as well as those inherent therein. The examples that follow are descriptions of embodiments and are not intended as limitations on the scope of the invention. Modifications therein and other uses will occur to those skilled in the art. These modifications are encompassed within the spirit of the invention and are defined by the scope of the claims. Varying substitutions and modifications may be made to the invention disclosed herein without departing from the scope and spirit of the invention.

The invention illustratively described herein may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be made by those skilled in the art, and that such modifications and variations are considered to be within the scope of this invention as defined by the appended claims.

### Example 1

### Detection of DNA Fragments by PicoGreen

General assay conditions and considerations are described. However, for subsequent examples provided below, assay conditions were modified to test variables and to accommodate the experimental purpose and do not necessary limit the invention to specific embodiments.

HL-80 is a human AML cell line commercially available from ATCC (ATCC Cat #CCL-240™). Complete cell culture medium was prepared as follows: 100 ml heat-inactivated fetal bovine serum, 20 mL 1-M HEPES (pH 7.5), 10 ml. Penicillin/Streptomycin stock solution (see Table 1) was added to a 1-liter RPMI-1640 Medium. After mixing thoroughly, the complete medium is filtered through a 0.22-μm sterilized filtration apparatus (Nalgene). Incubation medium was prepared as follows: 5 mL Penicillin/Streptomycin stock solution and 25-ml heat-inactivated fetal bovine serum were mixed with 500-ml RPMI-1640 (w/o phenol-red and L-glutamine).

The general experimental procedure Was performed with the following protocol. Cells were cultured four to live days before compound treatment. Cell viability should be approximately greater than 92%. Cell density was counted and viability confirmed using the GUAVA PCA with Viacount 2.12 program. Cells were aliquoted and centrifuged at 300×g for 6 minutes. The supernatant was discarded and the cell pellet was resuspended to 0.15 million cells/mL with RPMI (phenol Red-Free) with 5% FBS and 1% Penicillin/Streptomycin. An aliquot of 40 uL of cell suspension was dispensed to each well of a 384-well. Cells were incubated for the appropriate time under particular experimental conditions. Then, 45 uL of cell lysis buffer added to the cell samples.

Lysis buffer was prepared as follows: to make 1-liter of lysis buffer, 20 mL of 1-M Tris-HCl (pH 8.0) solution, 40 mL of 0.5-M EDTA (pH 8.0), 10 mL of 20% Tween-20 solution, 10 mL of 20% Triton X-100 solution are mixed with 920 mL deionized water. Just before use, 5 ml of an RNase A stock solution (10 mg/mL) was added into the lysis buffer to a final concentration of 0.05 mg/mL. After the addition of lysis buffer, the plates are allowed to stand at room temperature for 60 minutes. The cell culture plates were centrifuged at 2000×g for 20 min and 10 uL of the supernatant of the cell lysates containing the DNA fragments was transferred with a CyBio-well 384 into the detection plates (Corning Costar 384-well Polystyrene assay plate, black, non-binding surface). An aliquot of 10 uL of PicoGreen detection solution Was added to each sample well in the detection plates. The PicoGreen detection solution is made fresh before use by diluting the DMSO stock solution 1:200 into the detection buffer. The detection buffer is prepared by mixing 50 mL 10× Tris-HCL buffered saline (TES, pH 8.0) and 2 mL 0.5-M EDTA (pH 8.0) stock solutions with deionized water to final volume of 500 mL. Fluorescence intensity was analyzed with PerkinElmer Envision.

Table 1 lists non-limiting exemplary reagents and materials, concentrations, functions and supplier source.

TABLE 1

| Reagent/Plate | MW or Concentration | | Supplier, Cat. # | Function |
|---|---|---|---|---|
| RPMI 1640 | | | Gibco/BRL, 11875-085 | Cell culture medium |
| RPMI 1640 (phenol-red | | | Gibco/BRL, 11835-030 | Cell culture medium |
| free) | | | | |
| Dulbecco's Phosphate | 1x | | Gibco/BRL 14040-133 | Compound dilution buffer |
| buffered saline (DPBS) | | | | |
| Penicillin/Streptomycin | 10000 U/ml penicillin G | | Gibco/BRL, 15070-063 | antibiotics |
| (P/S) | 10000 ug/ml streptomycin | | | |
| | sulfate in 0.85% saline | | | |
| HEPES SOLUTION | 1 | M | Gibco/BRL, 15530-080 | buffer |
| FBS (Heat-inactivated) | | | Gibco/BRL, 16140-071 | Cell culture component |
| Plasmocin treatment | 50 | mg/ml | Invivogen ant-mpt | Antibiotics |
| DMSO | 100% | | Fisher Scientific | solvent |
| Camptothecin | 348.4 | | Sigma-Aldrich, C9911 | Reference compound |
| Vinblastine Sulfate | 909.1 | | Sigma-Aldrich, V1377 | Reference compound |
| EDTA | 0.5 | M (pH 8.0) | Fisher Scientific | DNase inhibitor |
| Tween-20 | 100% | | Fisher Scientific | detergent |
| Triton X-100 | 100% | | Fisher Scientific | detergent |
| RNase A | | | Sigma-Aldrich, R5500 | RNAdegradation enzyme |
| DNase-free RNase High | 10 | mg/mL | Roche Applied | RNA degradation enzyme |
| concentration. | | | Sciences, 1579681 | |
| Tris-buffered saline | 10x | | BioRad, 170-6435 | buffer |
| PicoGreen dye | 200x | | Molecular Probes, | Detection dye |
| | | | P7581 | |
| RQ1 RNase-Free DNase | 1 | Unit/μL | Fisher Scientific, | DNA degradationenzyme |
| | | | BP3223-1 | |

| | | |
|---|---|---|
| Cell Death Detection | Roche Applied | Nucleosome DNA |
| ELISA^{plus} kit | Sciences, 1774425 | detection |

Table 2 lists non-limiting examples of equipment, how such equipment can be used and a supplier.

TABLE 2

| Equipment | Supplier | Use |
|---|---|---|
| MatrixCellmate with | TiterTek | Plating cells |
| Stackers | | |
| CyBi-Well 384/1536 | CyBio | Compound library addition |
| | | and solution transfers |
| FlexiDrop | Perkin Elmer | Reagent Loading |
| Personal Cell Analysis | Guava | Cell counting and |
| | Technologies | viability tracking |
| Envision Multiplate Reader | Perkin Elmer | Fluorescence detector |

Measurement parameters on PerkinElmer Envision were as follows: for excitation, the mirror is FITC; excitation filter is FITC 485; emission filter is FITC 535; number of flashes equals 25; excitation light is 1%; detection gain is equal to 1 and measurement height is 8 mm.

Each assay contains positive, negative and blank controls. The appropriate controls used were determined by the experimental purposes to be achieved. Typically, the positive control was HL-60 cells treated with 5 uM vinblastine sulfate in 1% DMSO. The negative control was HL-60 cells treated with 1% DMSO. The signal blank was incubation medium with 1% DMSO no (HL-60 cells).

Data analysis can be adjusted to the experimental parameters or the paradigm under investigation. Typically, the relative amount of fragmented DNA formed was represented by the fluorescence intensity (FI) of a sample. The effect of an agent treatment on DNA fragmentation in HL-60 cells was calculated based on the change in fluorescence intensity relative to the DMSO control samples. Percent effect was determined as:

% Effect=100*[(FI_{agent}−mean(FI_{negative control}))/mean(FI_{negative control})]

### Example 2

### PicoGreen Specifically Detects DNA Fragments Released in HL-60 Cells

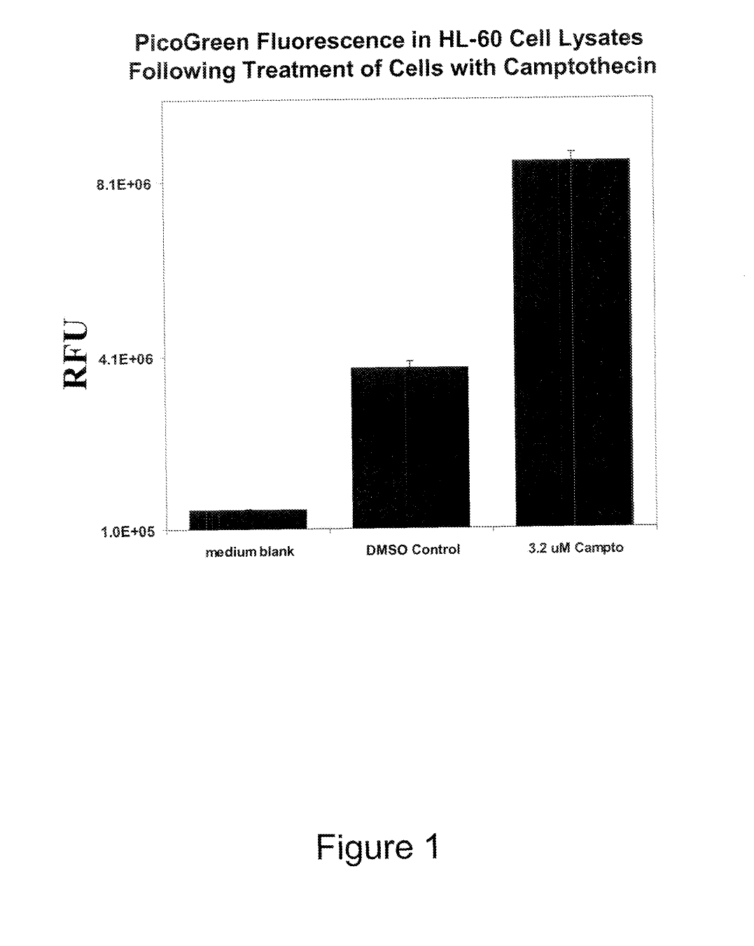

FIG. 1 shows PicoGreen fluorescence intensity increased in HL-60 treated with camptothecin. HL-60 cells in mid-log phase (0.4 million cells/mL) were treated with either 0.1% DMSO carrier solvent or 3.2 uM camptothecin for 5.5 hours. An equal volume of the lysis buffer (20 mM Tris-HCl (pH 8.0), 20 mM EDTA, 0.2% Tween-20) was added into the total cell culture. After standing at room temperature for 45 minutes, the cell lysate was subjected to centrifugation at 2000×g for 20 min and the top portion of the supernatant was withdrawn and DNA content was quantitated using fluorescence intensity readout by mixing with PicoGreen dye. Medium blank is the equal mixture of cell culture medium and lysis buffer.

### Example 3

### PicoGreen Fluorescence Signal is Dependent on the Level of DNA in the Cell Lysates

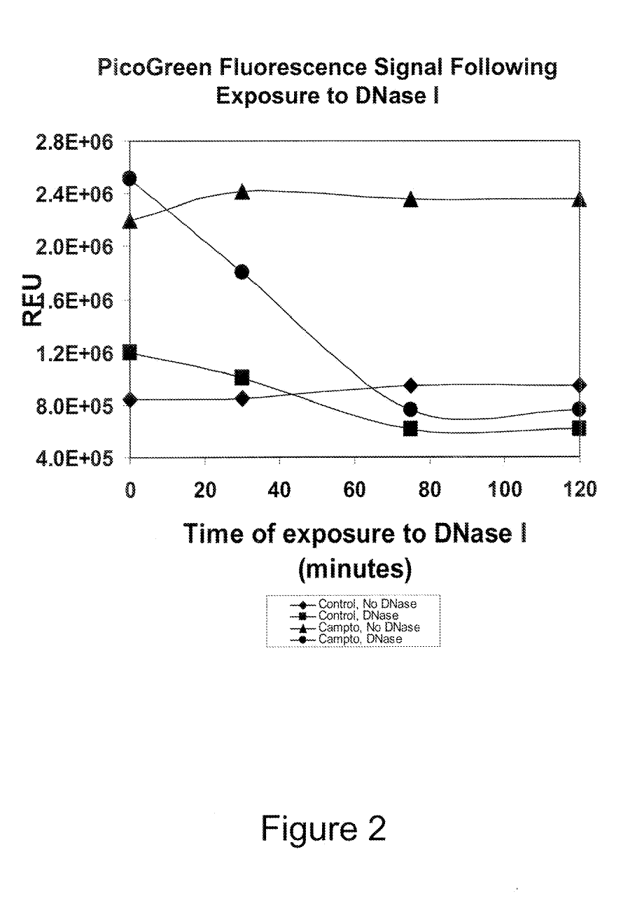

After treatment with either 0.1% DMSO carrier solvent or 3.2 uM camptothecin for 5.5 hours, an equal volume of the lysis buffer without EDTA (20 mM Tris-HCl (pH 8.0) 0.2% Tween-20 and 3 ug/mL RNase) was added into the HL-60 total cell cultures. After standing at room temperature for 45 minutes, the cell lysates were centrifuged and the top portion of the supernatant was withdrawn and incubated with RNase-free DNase at 37° C. for the indicated time (FIG. 2). After 2 hours of treatment, DNase I was able to reduce the fluorescence intensity of DMSO control sample by about 50%. The signal for camptothecin treated cells had higher fluorescence intensity before DNase treatment and was reduced effectively to a level similar to that of DMSO control with DNase I treatment. These results indicate that the PicoGreen fluorescent signal is due to the presence of fragmented DNA in the cell lysates.

### Example 4

### Comparison of PicoGreen to Propidium Iodide or ELISA for Detecting dsDNA

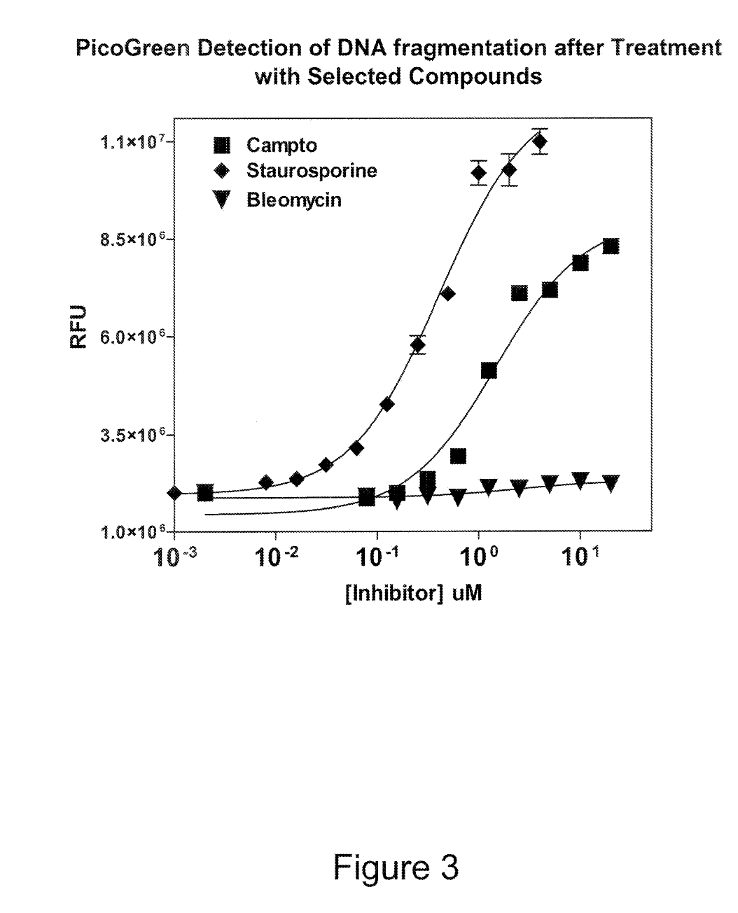

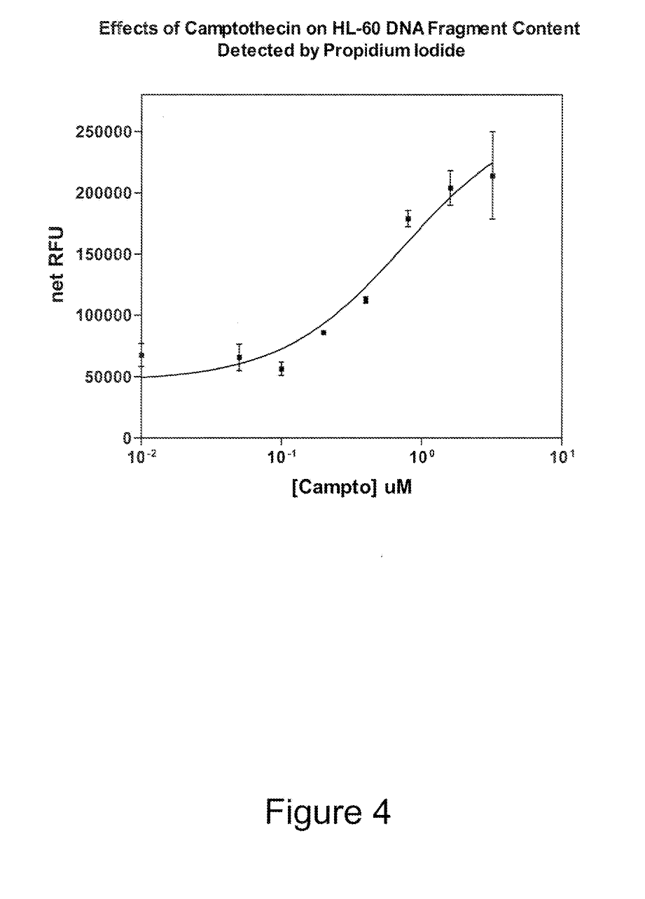

HL-60 cells in mid-log phase (0.3 million cells/ml) were treated with different doses of camptothecin, staurosporine or bleomycin for 20 hours. An equal volume of the lysis buffer (20 mM Tris-HCl (pH 8.0), 20 mM EDTA, 0.2% Tween-20 and 5 ug/mL RNase) was added into the total cell culture. The cell lysate was centrifuged at 2000×g for 20 min and top portion of the supernatant was withdrawn and DNA content was quantitated using either an ELISA kit (Roche Applied Sciences) or fluorescence intensity readout using PicoGreen or propidium iodide. The dose response curve of camptothecin using PicoGreen detection (FIG. 3) was compared to propidium iodide detection (FIG. 4). Propidium iodide was diluted from a 0.5 mg/ml stock to 0.00125 mg/mL working solution in 10 mM Tris-HCL (pH 7.5) with 1 mM EDTA. 20 uL of the propidium iodide working solution was mixed with 20 uL of sample solution before measurement of fluorescence intensity on PerkinElmer Envision with excitation wavelength: 531 nm; emission wavelength 635 nm.

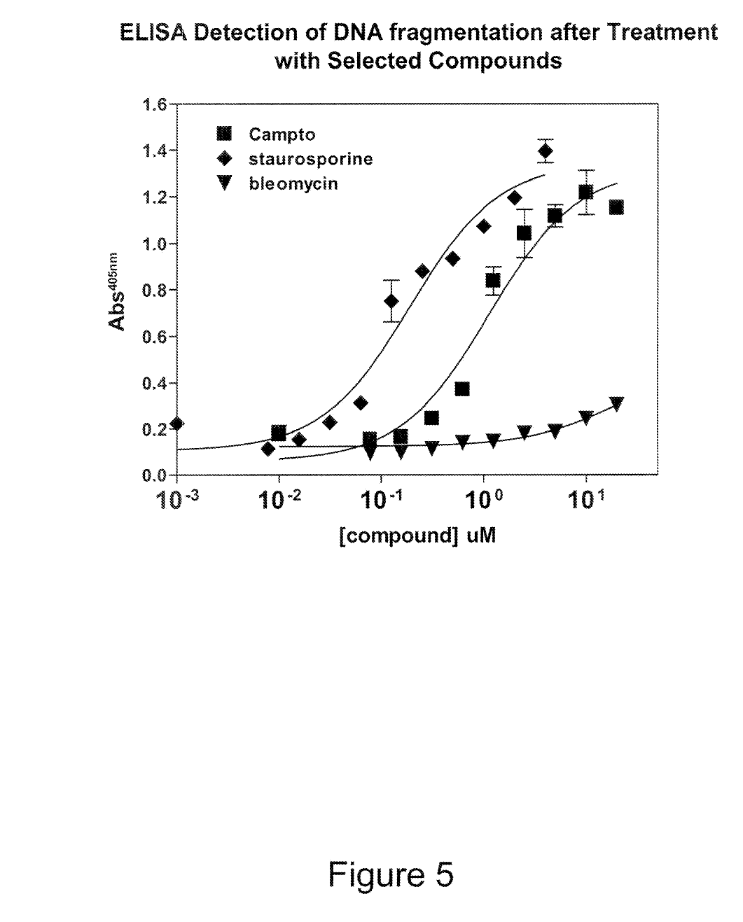

The dose response curves for camptothecin, staurosporine or bleomycin detected with PicoGreen are shown in FIG. 3. The EC₅₀ value for camptothecin was 1.48 uM, staurosporine was 0.41 uM and Neomycin was greater than 100 uM. The EC₅₀ values as determined by PicoGreen were in good agreement with the ELISA detection kit (FIG. 5). The EC₅₀ values determined by ELISA were 1.11 uM for camptothecin, 0.19 uM for staurosporine and greater than 100 uM for Neomycin.

### Example 5

### Effect of ZnCl₂, an Apoptosis Inhibitor, on Camptothecin-Induced DNA Fragmentation in HL-60 Cells

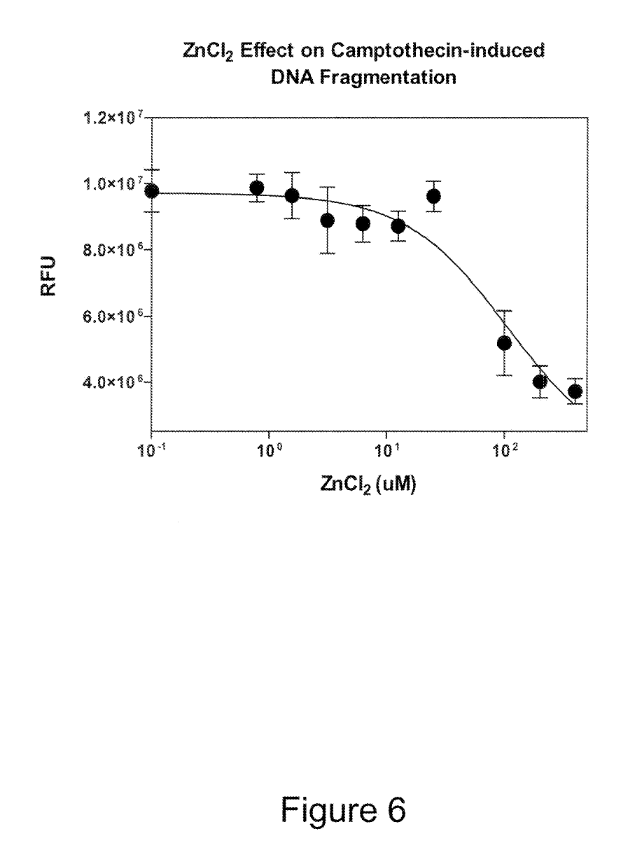

Zinc has been known to inhibit apoptosis induced by both chemical and death-receptor agonists. To further demonstrate the feasibility of applying the PicoGreen assay to detect and quantify dsDNA as a measure of DNA fragmentation in apoptosis, HL-60 cells in mid-leg phase (0.3 million cells/mL) were treated with 3.2 μM camptothecin in the presence of different doses of zinc chloride for 20 hours (FIG. 6). DNA fragments released from cells were quantified with PicoGreen reagent as described above. For samples with camptothecin, without zinc chloride or at low concentrations, fluorescence signal was more than 3 fold of that from samples with DMSO treatment. When zinc chloride concentrations were increased to 100 uM or higher, the magnitude of DNA fragmentation, which was reflected by the level fluorescent signal, decreased to the same level as samples with DMSO treatment only.

### Example 6

### Effects of RNase Treatment on DNA Fragmentation Signal to Background Ratio in HL-60 Lysates

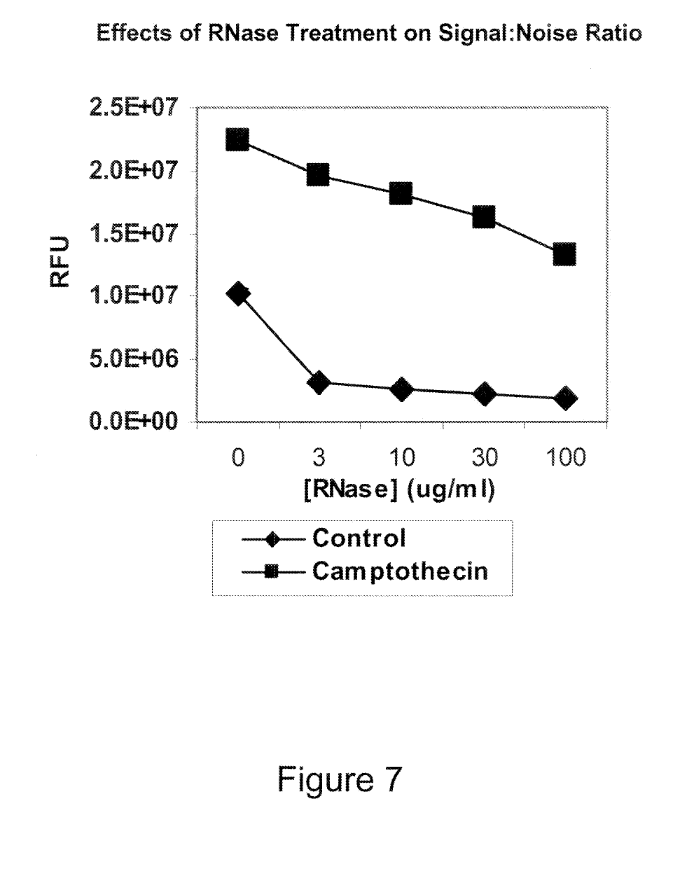

HL-60 cells in mid-log phase (0.4 million cells/mL) were treated with either 0.1% DMSO carrier solvent or 3.2 uM camptothecin for 5.5 hours. An equal volume of the lysis buffer (20 mM Tris-HCl (pH 8.0), 20 mM EDTA, 0.2% Tween-20) with different concentrations of DNase-free RNase (Roche Applied Sciences 1579681) were added into the total cell culture. After standing at room temperature for 45 minutes, the cell lysate was subjected to centrifuge at 2000×g for 20 min. The top portion of the supernatant was withdrawn and DNA content was quantitated by mixing with PicoGreen dye. Treatment of the cell lysate with high concentrations of RNase decreased background fluorescence due to cellular RNA and improved the signal window (FIG. 7).

### Example 7

### Time Course of Camptothecin Effects on DNA Fragmentation in HL-60 Cells

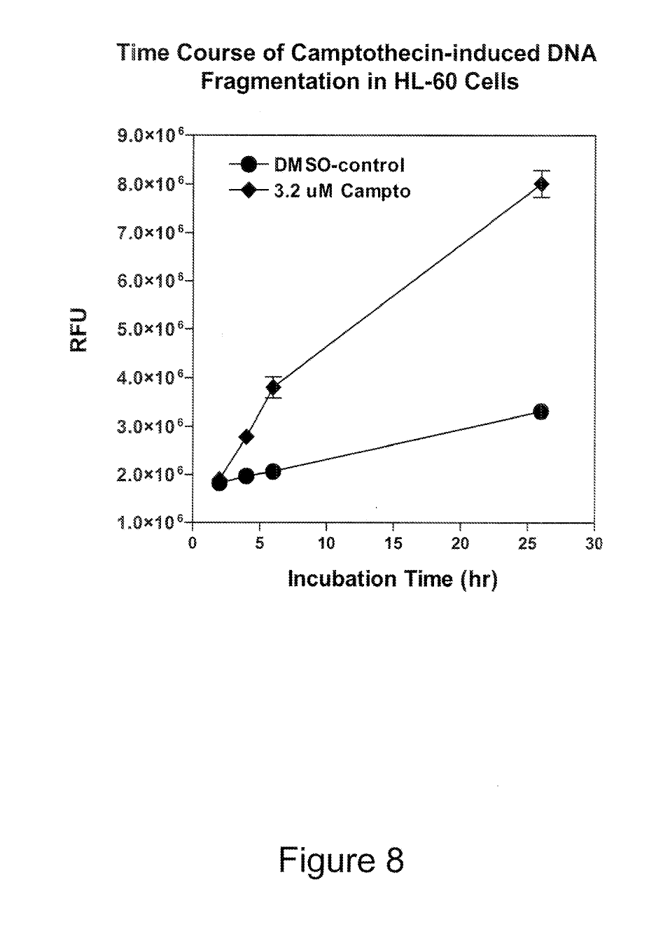

HL-80 cells in mid-log phase (0.4 million cells/mL) were treated with either 0.1% DMSO carrier solvent or 3.2 uM camptothecin (FIG. 8). At each time point indicated, 100 uL of the cell suspension was withdrawn to mix with equal volume of the lysis buffer (20 mM Tris-HCl (pH 8.0), 20 mM EDTA, 0.2% Tween-20). Camptothecin is a fast acting apoptosis-inducing agent. At 4 hours of treatment, camptothecin already caused a significant increase in DNA fragmentation.

### Example 8

### Effects of Cell Density on DNA Fragmentation in HL-60 Cells

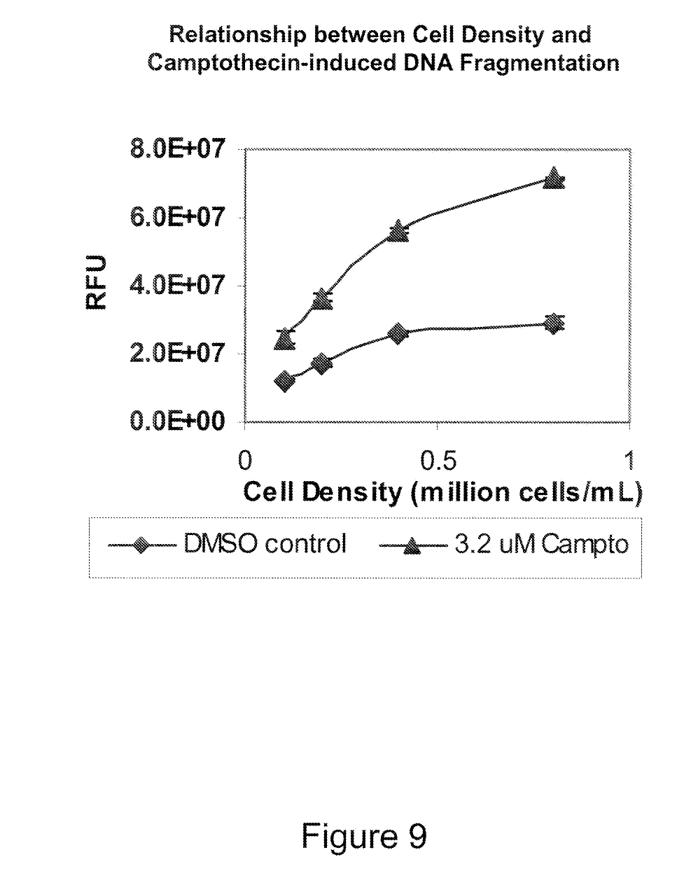

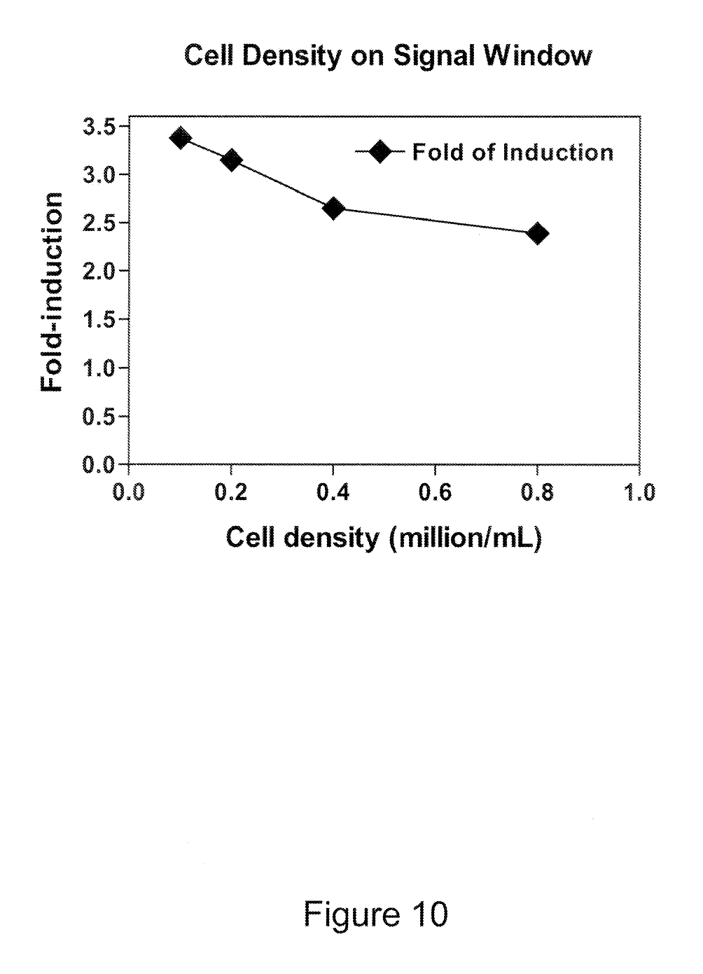

HL-60 cells in cell culture medium were spun down at 300×g for 6 min. After discarding the medium, cells were re-suspended into compound incubation medium to indicated concentration. The cell suspensions were then incubated with 1/10 volume of either 0.1% DMSO carrier solvent or 3.2 uM camptothecin for 20 hours before lysis and detection procedure. Medium blank is the equal mixture of cell culture medium and lysis buffer, FIG. 9 shows the effect of cell density on the signal window and FIG. 10 graphically represents the fold-induction in signal relative to cell density.

### Example 9

### DMSO Tolerance in HL-60 Cells

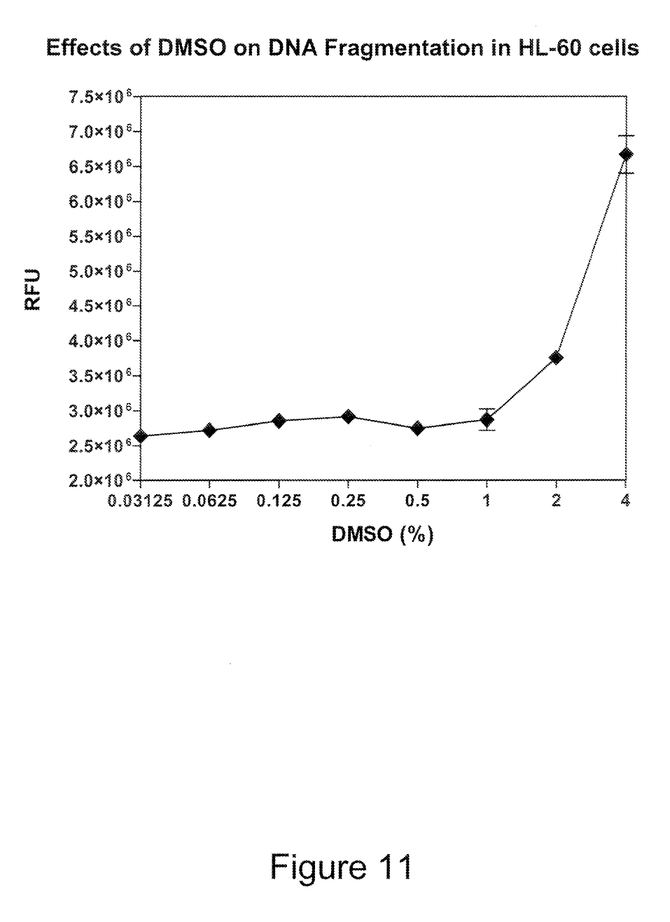

HL-60 cells in mid-log phase (0.3 million cells/mL) were treated with different doses of DMSO for 20 hours. An equal volume of the lysis buffer (20 mM Tris-HCl (pH 8.0), 20 mM EDTA, 0.2% Tween-20 and 5 ug/ml. RNase) was added into the total cell culture. The cell lysate was centrifuged at 2000×g for 20 min. The top portion of the supernatant was withdrawn and DNA content was quantitated using the PicoGreen fluorescent assay. FIG. 11 shows that up to 1% DMSO could be tolerated by HL-60 cells for 20 hr incubation.

### Example 10

### Dose Response Curves of a Panel of Cytotoxic Agents with Different Mechanisms of Action

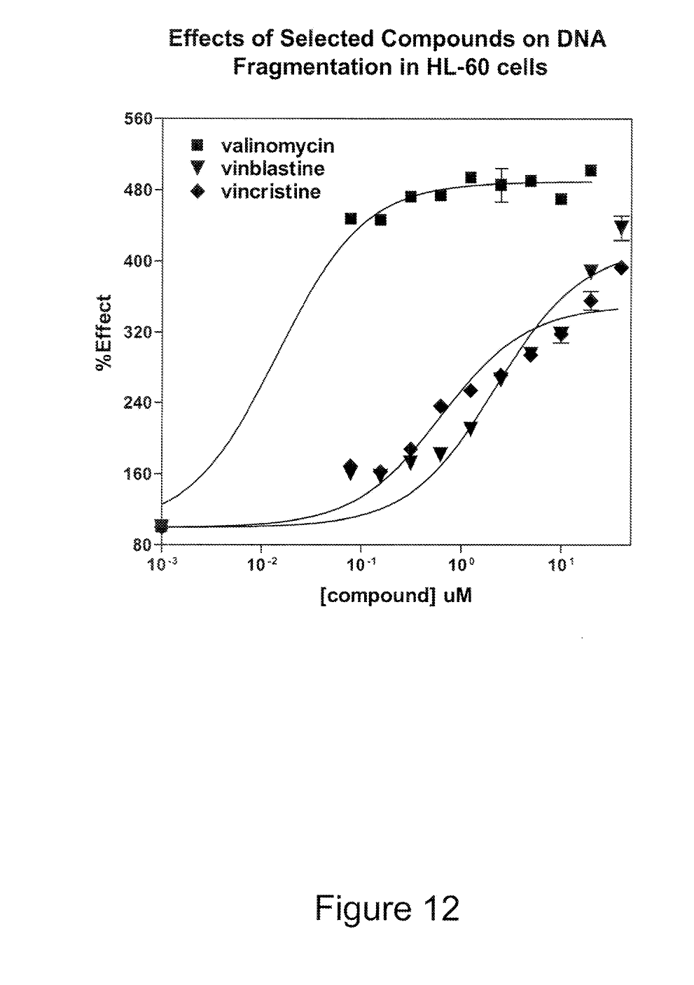

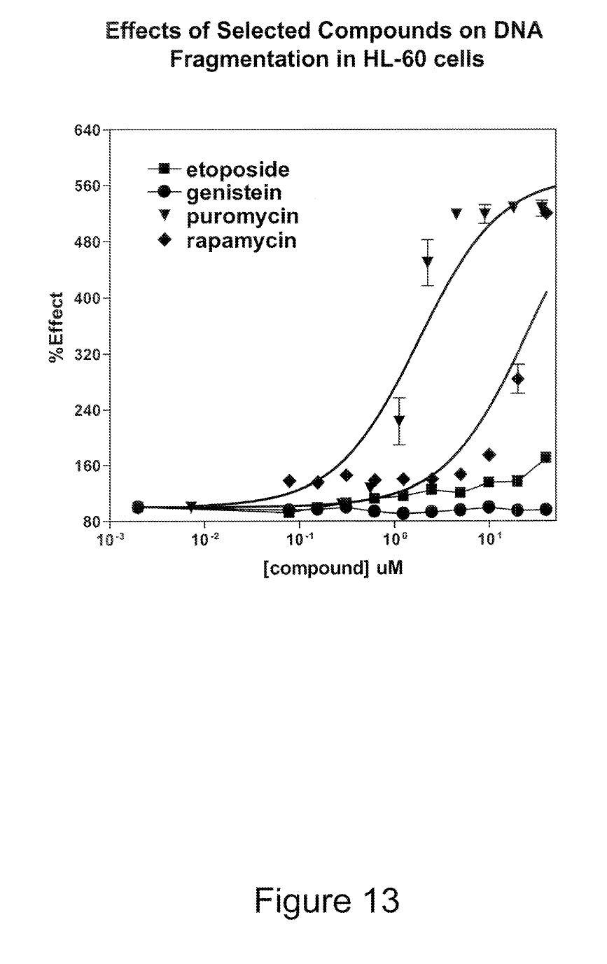

HL-60 cells in mid-log phase (0.3 million cells/mL) were treated with different doses of known apoptosis inducing compounds for 20 hours. FIG. 12 shows the cytotoxic activity of valinomycin, vinblastine and vincristine. FIG. 13 shows the cytotoxic activity of etoposide, genistein, puromycin and rapamycin.

### Example 11

### Data Distribution of Screening a Random Chemical Library

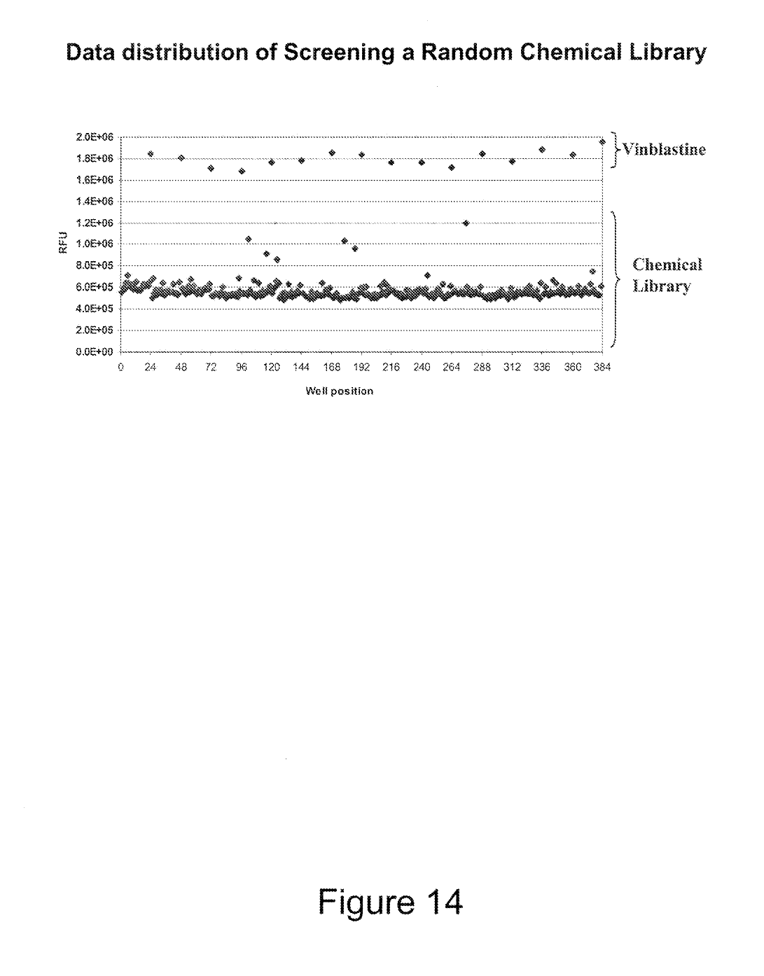

The compound library was dispensed to a 384 well into wells from column 1 to 22. The positive control, vinblastin (5 uM) was added to wells in column 24. Wells in column 23 were used for the negative control without compound). HL-60 cells were aliquoted to each well and were incubated for 40 hours (FIG. 14). DNA fragmentation was measured using the procedure described in Example 1.

What is claimed is:

1. A method of identifying an agent that modifies the formation of DNA fragments, the method comprising:
a) providing cells in an array of receptacles;
b) adding an agent to at least one receptacle;
c) incubating the agent with the cells for a predetermined period of time;
d) lysing the cells;
e) adding a detectable compound capable of intercalating into DNA fragments to said at least one receptacle;
f) measuring the amount of detectable compound intercalated; and
g) comparing the amount of intercalated detectable compound to a control to determine a difference
thereby identifying said agent as a modifying agent when the difference exceeds a predetermined threshold.

2. A method of identifying an agent that modifies the formation of DNA fragments, the method comprising:
a) providing cells in an array of receptacles;
b) adding to at least one receptacle a component selected from the group consisting of an inducer, an inhibitor, a modulator, a modulator of the inducer and a modulator of the inhibitor;
c) incubating the component with the cells for a predetermined period of time;
d) adding an agent to said at least one receptacle;
e) incubating the agent with the cells for a predetermined period of time;
f) lysing the cells;
g) adding a detectable compound capable of intercalating into DNA fragments to said at least one receptacle;
h) measuring the amount of detectable compound intercalated; and
i) comparing the amount of intercalated detectable compound to a control to determine a difference
thereby identifying said agent as a modifying agent when the difference exceeds a predetermined threshold.

3. The method of claim 2 wherein step (d) is combined with step (b) and step (e) is combined with step (c).

4. The method of claim 1 or 2 wherein chromosomal DNA is separated from DNA fragments before measuring the amount of detectable compound intercalated.

5. The method of claim 4 wherein the chromosomal DNA is separated from the double-stranded DNA fragments by a process selected from the group consisting of centrifugation, filtration, sedimentation, electrophoresis, size-exclusion, precipitation and affinity purification.

6. The method of claim 1 or 2 wherein the detectable compound comprises a substance selected from the group consisting of a radioactive isotope, a chemical that fluoresces, a peptide tag, a scintillant-activating compound, an enzyme and an epitope recognized by a detectable antibody.

7. The method of claim 6 wherein the detectable compound comprises a substance selected from the group consisting of PicoGreen, SYBR Green, TOTO, YOPRO, BENA435, Hoechst 33258, Hoechst 33342, DAPI, DRAQ5, OliGreen and propidium iodide.

8. The method of claim 1 or 2 wherein the cell comprises a prokaryotic cell.

9. The method of claim 1 or 2 wherein the cell comprises a eukaryotic cell.

10. The method of claim 1 or 2 wherein the cell is transiently or stably transformed to overexpress at least one protein.

11. The method of claim 1 or 2 wherein the cell is provided following isolation from a biological sample.

12. The method of claim 11 wherein the biological sample is from a human.

13. The method of claim 1 or 2 wherein the cell is an HL60 cell.

14. The method of claim 1 or 2 wherein one or more steps are performed by a robotic device.

15. The method of claim 1 or 2 wherein the cells are lysed by a process selected from the group consisting of a lysis buffer containing a detergent, a hypotonic lysis buffer, sonication and freeze/thaw.

16. The method of claim 1 wherein RNAse is added during step (d) or step (e).

17. The method of claim 2 wherein RNAse is added during step (f) or step (g).

18. A method of identifying an agent that modifies the formation of DNA fragments, the method comprising
a) providing cells in an array of receptacles;
b) adding an agent to at least one receptacle;
c) incubating the agent with the cells for a predetermined period of time;
d) adding a detectable compound capable of intercalating into DNA fragments to said at least one receptacle;
e) measuring the amount of detectable compound intercalated; and
f) comparing the amount of intercalated detectable compound to a control to determine a difference
thereby identifying said agent as a modifying agent when the difference exceeds a predetermined threshold.

19. A method of identifying an agent that modifies the formation of DNA fragments, the method comprising:
a) providing cells in an array of receptacles;
b) adding to at least one receptacle a component selected from the group consisting of an inducer, an inhibitor, a modulator, a modulator of the inducer and a modulator of the inhibitor;
c) incubating the component with the cells for a predetermined period of time;
d) adding an agent to said at least one receptacle;
e) incubating the agent with the cells for a predetermined period of time;
f) adding a detectable compound capable of intercalating into DNA fragments to said at least one receptacle;
g) measuring the amount of detectable compound intercalated; and
h) comparing the amount of intercalated detectable compound to a control to determine a difference
thereby identifying said agent as a modifying agent when the difference exceeds a predetermined threshold.

20. The method of claim 1 further comprising providing a second array of receptacles wherein step (d) further comprises separating supernatant from cell debris and step (e) further comprises adding a detectable compound capable of intercalating into DNA fragments to at least one receptacle of said second array of receptacles containing a sample of said separated supernatant.

21. The method of claim 2 further comprising providing a second array of receptacles wherein step (f) further comprises separating supernatant from cell debris and step (g) further comprises adding a detectable compound capable of intercalating into DNA fragments to at least one receptacle of said second array of receptacles containing a sample of said separated supernatant.

22. An assay system for identifying an agent that modifies the formation of DNA fragments, the assay system comprising:
a) an array of receptacles;
b) a lysis buffer;
c) a detectable compound capable of intercalating into DNA; and
d) at least one component wherein the component is selected from the group consisting of the agent(s), an inducer(s), an inhibitor, a modulator(s), a modulator(s) of the inducer(s), a modulator(s) of the inhibitor(s), control(s) and cells.

23. A kit comprising at least one element of the assay system of claim 20-22 and instructions for use.

24. A method of diagnosing or monitoring a treatment of a disease wherein a biomarker for the disease comprises the formation of DNA fragments, the method comprising:
a) providing a biological sample in an array of receptacles;
b) adding a detectable compound capable of intercalating into DNA fragments to at least one receptacle;
c) measuring the amount of detectable compound intercalated; and
d) comparing the amount of intercalated detectable compound to a reference to determine a difference
thereby diagnosing or monitoring the treatment of the disease when the difference exceeds a predetermined threshold.

25. The method of claim 24 wherein the biological sample comprises a sample selected from the group consisting of cells, tissues, organs, and blood.

26. An assay system for diagnosing or monitoring a treatment of a disease wherein a biomarker for the disease comprises the formation of DNA fragments, the assay system comprising:
a) an array of receptacles;
b) a detectable compound capable of intercalating into DNA; and
d) at least one control.
